SEQUENCE LISTING (30 sequences)

SEQ ID NO: 1 - protein, 120 aa, Mus musculus
QVQLKQSGAELVRPGASVRLSCKASGYTFTFYWINWIKQRPEQGLEWIGRIDPYDSETRYNQKFKDKAILTVDKYSSTAYMQLSSLTSEDSAVYYCAKGVYDGHWFFDVWGAGTSVTVSS

SEQ ID NO: 2 - protein, 108 aa, Mus musculus
DIVMTQSHKFMSTSVGDRVSITCKASQDVSTAVAWYQQKPGQSPKLLIYSASYRYTGVPARFSGSGSGTDFTFTISSVQTEDLAVYYCQQHYRTPPTFGGGTKLELKR

SEQ ID NO: 3 - protein, 119 aa, Mus musculus
QVQLQQSGPELEKPGASVKISCKASGYSFSDYNMNWVKQSNGKSLEWIGNIDPYNGDTNYNQKFKGKATLTLDKSSSTAYMQLKSLTSEDSAVYFCARSRGWLLPFAYWGQGTLVTVSA

SEQ ID NO: 4 - protein, 108 aa, Mus musculus
DILMTQSQKFMSTSVGDRVSVTCKASQNVGINVAWYQQKPGQSPKALIYSASYRYSGVPDRFTGSGSGTDFTLTISNVQSEDLAEYFCQQYNSYPLTFGGGTKLEIKR

SEQ ID NO: 5 - protein, 117 aa, Mus musculus
QVQLQQSGPELVRPGVSVKISCKGSGYTFTDYAIHWVRQSHAQSLEWIGVIGTYSGNRNYNQKFKGKATMTVDKSSSTAYMELARLTSEDSAIYYCARSAGYSLDFWGQGTSVTVSS

SEQ ID NO: 6 - protein, 112 aa, Mus musculus
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGKHFPWTFGGGTKLEIK

SEQ ID NO: 7 - protein, 119 aa, Mus musculus
QVQLQQSGGELVRPGTSVKVSCKASGYAFTNYLIEWIRQRPGQGLEWIGVINPGSGNSKSSKNLKGKATLTADKSSNTAYMQLSSLTSDDSAVYFCARSGVYGSSPDYWGQGTTLTVSS

SEQ ID NO: 8 - protein, 113 aa, Mus musculus
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIKR

SEQ ID NO: 9 - protein, 118 aa, Mus musculus
QVQLQQSGGELVRPGTSVKVSCKASGYAFTNYLIEWVKQRPGQGLDWIGMINPGSGGTKCNKKFKGKVTLTADKSSSTAYMHLSSLTSDDSAVYFCARSGWVSAMDYWGQGTSVTVSS

SEQ ID NO: 10 - protein, 113 aa, Mus musculus
DIVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLELKR

SEQ ID NO: 11 - protein, 118 aa, Mus musculus
QVQLQQSGVELVRPGTSVKVSCKASGYAFTNYLIEWVKQRPGQGLDWIGMINPGSGGTKCNKKFKGKVTLTADKSSSTAYMHLSSLTSDDSAVYFCARSGWVYAMDYWGQGTSVTVSS

SEQ ID NO: 12 - protein, 113 aa, Mus musculus
DVLMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLELKR

SEQ ID NO: 13 - protein, 117 aa, Mus musculus
QVQLQQSGPELVRPGVSVKISCKGSGYRFTDYAIHWVKQSHAKSLEWIGVISTYYGNTRYNQKFKGKATMTVDKSSSTAYMELASLTSEDSVIYYCARSGGSLMDYWGQGTSVTVSS

SEQ ID NO: 14 - protein, 113 aa, Mus musculus
DIVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKLEIKR

SEQ ID NO: 15 - protein, 117 aa, Mus musculus
QVQLQQSGPELVRPGVSVKISCKGSGYTFTDYAMHWVKQSHAKSLEWIGVISTYYSNTRYNQKFKGKATMTVDKSSSTAYMELARLTSEDSAIYYCVRSGGSNMDYWGQGTSVTVSS

SEQ ID NO: 16 - protein, 113 aa, Mus musculus
DIQMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPWTFGGGTKLEIKR

SEQ ID NO: 17 - protein, 117 aa, Mus musculus
QVQLQQSGPELVRPGVSVKISCKGSSYKFTDYAMHWVKQSHAKSLEWIGVISTYYGNVKYNQKFKGKATMTVDKSSSTAYMELARLTSEDSAVYYCARSSGSYLDYWGQGTSVTVSS

SEQ ID NO: 18 - protein, 113 aa, Mus musculus; identical to SEQ ID NO: 14

SEQ ID NO: 19 - protein, 119 aa, Mus musculus
QVQLQQSGAELVRPGTSVKVSCKASGYAFTNYLIEWVKQRPGQGPEWIGVINPGSGNIRYNGKFKGKATLTADKSSSTAYMQLSSLTSDDSAVYFCARDAYYVGAMDYWGQGTSVTVSS

SEQ ID NO: 20 - protein, 113 aa, Mus musculus
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLELKR

SEQ ID NO: 21 - protein, 119 aa, Mus musculus
QVQLQQSEAELVRPETSVKVSCKASGYSFRNYLIEWVKQRPGQGLEWIGVINPGSGNTKYNEKFKGKATLTADKSSSTAYMQLSSLTSDDSAVYFCARDGYYLGTMDYWGQGTSVTVSS

SEQ ID NO: 22 - protein, 113 aa, Mus musculus
DIVLTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKLEIKR

SEQ ID NO: 23 - protein, 119 aa, Mus musculus
QVQLQQSGAELVRPGTSVKVSCKASGYAFINNLIEWVQQRPGQGLEWIGVINPGSGNVKYNEKFKGKATLTADKSSSTAYMQLSSLTSDDSAVYFCARDGYYLGTMDHWGQGTSVTVSS

SEQ ID NO: 24 - protein, 113 aa, Mus musculus
DVVMTQTPLTLSVTIGQPASISCKSSQSLLDSDGKTYLNWLLQRPGQSPKRLIYLVSKLDSGVPDRFTGSGSGTDFTLKISRVEAEDLGIYYCWQGTHFPWTFGGGTKLELKR

SEQ ID NO: 25 - protein, 117 aa, Mus musculus
EVQLQQSGPELVRPGVSVKISCKGSSYKFTDYAMHWVKQSHAKSLEWIGVISTYYGNVKYNQKFKGKATMTVDKSSSTAYMELARLTSEDSAVYYCARSYGSYLDYWGQGTSVTVSS

SEQ ID NO: 26 - protein, 112 aa, Mus musculus
DIVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPWTFGGGTKLEIK

SEQ ID NO: 27 - DNA, 22 bp, Artificial
  an artificially synthesized primer sequence
cagctatgaaatacctattgcc

SEQ ID NO: 28 - DNA, 23 bp, Artificial
  an artificially synthesized primer sequence
cttttcataatcaaaatcaccgg

SEQ ID NO: 29 - DNA, 19 bp, Artificial
  an artificially synthesized primer sequence
attgcctacggcagccgct

SEQ ID NO: 30 - DNA, 20 bp, Artificial
  an artificially synthesized primer sequence
aaatcaccggaaccagagcc

(19) United States
(12) Patent Application Publication
(10) Pub. No.: US 2006/0204496 A1
(43) Pub. Date: Sep. 14, 2006

(54) Agonist antibody against heteroreceptor

(76) Inventors: Tetsuo Kojima, Shizuoka (JP); Chiaki Senoo, Shizuoka (JP); Osamu Natori, Shizuoka (JP); Keiko Kasutani, Shizuoka (JP); Shiny A Ishi, Shizuoka (JP)

Correspondence Address: FISH & RICHARDSON PC, P.O. BOX 1022, MINNEAPOLIS, MN, 55440-1022, US

(21) Appl. No.: 10/540,643

(22) PCT Filed: Nov. 28, 2003
(86) PCT No.: PCT/JP03/15230

§ 371 Date: Mar. 10, 2006

(51) Int. Cl. A61K 39/395; C07K 16/28

(52) U.S. Cl. 424/143.1; 530/388.22

(57) ABSTRACT

Animals were immunized with either the A or B chain of a receptor, and then mRNA was extracted from the spleen cells of the animals, and the variable regions of the L and H chains were isolated by RT-PCR using primers for variable regions comprising CDRs. Single-chain Fv was synthesized by assembly PCR to construct a phage library. Clones for antigen-bound antibodies were concentrated and cloned by panning. An expression vector for scFv-CH1-Fc was prepared by inserting a single-chain variable region between CH1-hinge-CH2-CH3 and the signal sequence for animal cells. Various combinations of such expression vectors were introduced into cells to express antibodies, and antibody clones exhibiting ligand-like activity were selected.

### TECHNICAL FIELD

The present invention relates to antibodies to heteromeric receptors, and to pharmaceutical compositions comprising the antibodies as active ingredients.

### BACKGROUND ART

Since antibodies are highly stable in the blood and have no antigenicity, they are drawing much attention as pharmaceuticals. It has been a while since the proposal of bispecific antibodies, which can simultaneously recognize two types of antigens, however at present only those binding just two types of antigens exist. However, since antibodies bind to specific epitopes within antigens, it may be possible to place two antigens at desirable distances and angles by selecting appropriate antibody combinations.

In many cytokine receptors, the angle and distance apart of chains that form dimers is thought to change when a ligand binds, enabling transmission of signals into cells. Thus, appropriate anti-receptor antibodies can mimic the receptor dimerization initiated by ligand-binding, and can become agonistic antibodies. Previous reports describe monoclonal antibodies with agonist activity against MPL (see US Patent Application No. 98/17364 and “Blood” (1998) Vol. 92, No. 6, pp. 1981-1988)), EPO receptor, or GH receptor, which are each made up of homodimers.

However, when a receptor is made up of heterodimers, a complex of two different receptor chains must be formed, and thus ordinary antibodies cannot be predicted to have agonist activity. The bispecific antibodies described above, which can recognize two types of antigens with their two arms, may be suitable for this purpose, but there are no reports of such utility.

### DISCLOSURE OF THE INVENTION

The present invention was achieved in view of the above. An objective of the present invention is to provide antibodies having agonist activity to receptors comprising heterologous chains.

The present inventors conducted extensive studies to achieve the objective described above. To screen for agonist antibodies, many antibodies (α and β) against each of the two chains (A and B) forming the receptor must be selected, and various combinations of α and β must be tested one-by-one for agonist activity. Furthermore, to produce bispecific antibodies, it is necessary to fuse antibody-producing hybridomas, or to introduce antibody expression vectors into cells. The present inventors succeeded in preparing bispecific antibodies having agonist activity to receptors comprising heterologous chains by using the methods described below, for example. Specifically, the inventors used the procedure described below:

Animals were immunized with either an A or B chain of a receptor, mRNA was extracted from their spleen cells, and the variable regions of the L and H chains were isolated by RT-PCR using primers corresponding to the variable regions containing the antibody complementarity determining regions (CDRs). Single-chain Fv (scFv) were synthesized by assembly PCR. A phage library was constructed using the PCR products. Antigen-binding antibody clones were concentrated and cloned by panning, and the single-chain variable regions (scFv) were inserted between CH1-hinge-CH2-CH3 and the signal sequence for animal cells, to construct an expression vector for scFv-CH1-Fc. Various combinations of variable regions were introduced into cells, and the antibodies were expressed. The resulting culture supernatants were added to cells that responded to a ligand of interest, and antibody clones exhibiting ligand-like activity were selected.

By using the methods described above, the present inventors succeeded in isolating agonist antibodies to type-I interferon receptor, which comprises two types of chains: AR1 and AR2. Specifically, the present inventors succeeded for the first time in isolating bispecific antibodies having agonist activity against receptors comprising heterologous chains, thus completing the present invention.

The present invention relates to antibodies having agonist activity to receptors comprising heterologous chains. More specifically, the present invention provides:

[1] an antibody having agonist activity to a receptor comprising a heterologous chain;

[2] the antibody of [1], wherein the receptor is a cytokine receptor;

[3] the antibody of [2], wherein the cytokine receptor is an interferon receptor;

[4] the antibody of [3], wherein the interferon receptor is a type-I interferon receptor;

[5] the antibody of [4], wherein the type-I interferon receptor comprises an AR1 chain and an AR2 chain;

[6] the antibody of [1], wherein the receptor is a multimer;

[7] the antibody of [6], wherein the multimer is a dimer;

[8] the antibody of any one of [1] to [7], wherein the antibody is a bispecific antibody;

[9] the antibody of [5], comprising a variable region of an anti-AR1 chain antibody and a variable region of an anti-AR2 chain antibody;

[10] the antibody of [9], comprising a variable region of an anti-AR1 chain antibody that comprises the amino acid sequences of (a), and a variable region of an anti-AR2 chain antibody that comprises the amino acid sequences of any one of (b1) to (b10):

(a) the amino acid sequence of SEQ ID NO: 1 as the H chain variable region, and the amino acid sequence of SEQ ID NO: 2 as the L chain variable region;

(b1) the amino acid sequence of SEQ ID NO: 7 as the H chain variable region, and the amino acid sequence of SEQ ID NO: 8 as the L chain variable region;

(b2) the amino acid sequence of SEQ ID NO: 9 as the H chain variable region, and the amino acid sequence of SEQ ID NO: 10 as the L chain variable region;

(b3) the amino acid sequence of SEQ ID NO: 19 as the H chain variable region, and the amino acid sequence of SEQ ID NO: 20 as the L chain variable region;

(b4) the amino acid sequence of SEQ ID NO: 13 as the H chain variable region, and the amino acid sequence of SEQ ID NO: 14 as the L chain variable region;

(b5) the amino acid sequence of SEQ ID NO: 23 as the H chain variable region, and the amino acid sequence of SEQ ID NO: 24 as the L chain variable region;

(b6) the amino acid sequence of SEQ ID NO: 5 as the H chain variable region, and the amino acid sequence of SEQ ID NO: 6 as the L chain variable region;

(b7) the amino acid sequence of SEQ ID NO: 17 as the H chain variable region, and the amino acid sequence of SEQ ID NO: 18 as the L chain variable region;

(b8) the amino acid sequence of SEQ ID NO: 15 as the H chain variable region, and the amino acid sequence of SEQ ID NO: 16 as the L chain variable region;

(b9) the amino acid sequence of SEQ ID NO: 21 as the H chain variable region, and the amino acid sequence of SEQ ID NO: 22 as the L chain variable region;

(b10) the amino acid sequence of SEQ ID NO: 11 as the H chain variable region, and the amino acid sequence of SEQ ID NO: 12 as the L chain variable region;

[11] the antibody of [9], comprising the variable region of an anti-AR1 chain antibody that comprises the amino acid sequences of (a), and the variable region of an anti-AR2 chain antibody that comprises the amino acid sequences of any one of (b 1) to (b3):

(a) the amino acid sequence of SEQ ID NO: 3 as the H chain variable region, and the amino acid sequence of SEQ ID NO: 4 as the L chain variable region;

(b1) the amino acid sequence of SEQ ID NO: 9 as the H chain variable region, and the amino acid sequence of SEQ ID NO: 10 as the L chain variable region;

(b2) the amino acid sequence of SEQ ID NO: 25 as the H chain variable region, and the amino acid sequence of SEQ ID NO: 26 as the L chain variable region;

(b3) the amino acid sequence of SEQ ID NO: 21 as the H chain variable region, and the amino acid sequence of SEQ ID NO: 22 as the L chain variable region; and

[12] a pharmaceutical composition comprising the antibody of any one of [1] to [11] as an active ingredient.

The present invention provides antibodies having agonist activity to receptors comprising heterologous chains.

In the present invention, receptors comprising heterologous chains refer to receptors (multimers) made of two or more different proteins (receptor chains). Multimers include dimers, trimers, tetramers, etc. While there is no limitation on the number of proteins (receptor chains) constituting a receptor, dimers are preferred. For example, when receptors are dimers, heteromeric receptors have two non-identical protein components (receptor chains).

An antibody having agonist activity refers to an antibody having agonist function against a certain receptor. Typically, when an agonist ligand (factor) binds to a receptor, the tertiary structure of the receptor protein changes, and the receptor is activated (when the receptor is a membrane protein, a cell growth signal or such is usually transduced). If the receptor is a dimer-forming type, agonistic antibodies can dimerize the receptor at an appropriate distance and angle, thus acting similarly to a ligand. In other words, an appropriate anti-receptor antibody can mimic the receptor dimerization performed by a ligand, and thus can be used as an agonist antibody.

Physiological activities whose changes are induced by agonist function include, but are not limited to, growth activity, surviving activity, differentiation activity, transcriptional activity, membrane transport activity, binding activity, proteolytic activity, phosphorylation/dephosphorylation activity, oxidation/reduction activity, transfer activity, nucleolytic activity, dehydration activity, cell death-inducing activity, and apoptosis-inducing activity, for example.

In a preferred embodiment of the present invention, the receptors of the present invention include cytokine receptors. The term “cytokine” is typically used as a generic name for physiologically active proteins that regulate the proliferation and differentiation of various types of blood cells. However, it sometimes also refers to growth factors and growth-inhibiting factors for cells including non-immune cells. Thus, “cytokine” is a generic name for proteinous factors released from cells and mediating intercellular interactions, such as immune and inflammatory response regulation, antiviral activity, antitumor activity, and cell propagation/differentiation regulation.

Specific examples of cytokines are interleukins 1-15, colony-stimulating factors (G-CSF, M-CSF, GM-CSF, etc.), interferons (IFN-α, IFN-β, IFN-γ, etc.), chemokines, tumor necrosis factor (TNF), lymphotoxin, erythropoietin, epidermal growth factor, and fibroblast growth factor. Interferon is preferred, and type-I interferon is particularly preferred.

Specific examples of cytokine receptors are the interferon receptor family (IFN-α receptor, IFN-β receptor, IFN-γ receptor, IL-10 receptor, and such), the interleukin receptor family (IL-2 receptor, IL-3 receptor, IL-6 receptor, GM-CSF receptor, and such), the serine-threonine kinase receptor family (BMP receptor, TGF-β receptor, activin receptor, and such), the tyrosine kinase receptor family (EGF receptor, PDGF receptor, VEGF receptor, c-kit receptor, c-fms receptor, and such), the immunoglobulin receptor family (IL-1 receptor, and such), the receptor family associated with cell death (TNF receptor, Fas receptor, NGF receptor, and such), and the seven-transmembrane receptor family (IL-8 receptor, chemokine receptor, and such). Members of the interferon receptor family are preferred, and type-I interferon receptors are particularly preferred.

Interferons include IFN-α, IFN-β, IFN-γ, and IFN-τ. IFN-α and IFN-β are highly homologous, and therefore these two INFs can react through the same receptor. Interferon α and interferon β are categorized as type-I interferons.

Preferred type-I interferon receptors include, for example, receptors comprising an AR1 chain (GenBank accession No. J03171: Uze G; Lutfalla G, Gresser I., Genetic transfer of a functional human interferon [[alpha]] receptor into mouse cells: cloning and expression of its cDNA. Cell (1990) 60, 225-234.) and AR2 chain (GenBank accession No. U29584; Domanski P, Witte M, Kellum M, Rubinstein M, Hackett R, Pitha P, et al., Cloning and expression of a long form of the [[beta]] subunit of the interferon [[alpha]]/[[beta]] receptor that is required for signaling. J Biol Chem (1995) 270, 21606-21611; LutfaUa G; Holland S J, Cinato E, Monneron -D., Reboul J, Rogers N C, et al. Mutant U5A cells are complemented by an interferon-ab receptor subunit generated by alternative processing of a new member of a cytokine receptor gene cluster. EMBO J (1995) 14, 5100-5108.).

The term “multispecific antibody” refers to an antibody capable of binding specifically to multiple types of different antigens. In other words, a multispecific antibody is an antibody with specificity to at least two different antigens (for example, when the antigen is a heteromeric receptor, the multispecific antibody recognizes different domains that constitute the heteromeric receptor). Such a molecule typically binds to two antigens (a bispecific antibody), but may have specificity to more antigens (for example, three antigens).

There is no limitation on the type of antibody to a heteromeric receptor of the present invention, but monoclonal antibodies are preferred. Furthermore, recombinant antibodies produced by gene recombination techniques are preferred (see, for example, Borrebaeck, C. A. K. and Larrick, J. W., THERAPEUTIC MONOCLONAL ANTIBODIES, Published in the United Kingdom by MACMILLAN PUBLISHERS LTD, 1990). Such recombinant antibodies can be produced by cloning DNAs that encode antibodies from antibody-producing cells, such as hybridomas or sensitized lymphocytes that produce the antibody, then inserting the DNA into an appropriate vector, and introducing the resulting vector to a host.

Moreover, the antibodies of the present invention may be fragment thereof, modified antibodies, minibodies, or such. For example, antibody fragments include Fab, F(ab′)₂, and Fv. Herein, an “Fv” fragment is the smallest antibody fragment, and contains a complete antigen recognition site and a binding site. This region is a dimer (VH-VL dimer) wherein the variable regions of each of the heavy and light chain are strongly connected by a noncovalent bond. The three CDRs of each of the variable regions interact to form an antigen-binding site on the surface of the VH-VL dimer. Six CDRs form the antigen-binding site of an antibody. However, a variable region (or a half an Fv, containing only three CDRs specific to an antigen) alone also has the ability to recognize and bind an antigen, although its affinity is lower than that of the entire binding site.

A Fab fragment (also referred to as F(ab)) further includes a constant region of the light chain and a constant region (CH1) of the heavy chain. A Fab′ fragment differs from a Fab fragment in that it also has several residues derived from the carboxyl end of the heavy chain CH1 region which contains one or more cysteines from the hinge domain of the antibody. Fab′-SH indicates a Fab′ wherein one or more cysteine residues of the constant region have a free thiol-group. The F(ab′) fragment is produced by the cleavage of the disulfide bonds between the cystines in the hinge region of the F(ab′)2 pepsin digest. Other chemically bound antibody fragments are also known to those skilled in art.

Minibodies include single chain Fv (scFv), diabodies, linear antibodies, and single chain antibody molecules.

In the present invention, diabodies (Db) mean bivalent antibody fragments constructed by gene fusion (Holliger P. et al., Proc. Natl. Acad. Sci. USA 90: 6444-6448 (1993); EP 404,097; WO93/11161). Diabodies are dimers comprising two polypeptide chains, where each polypeptide chain comprises a light chain variable domain (VL) and a heavy chain variable domain (VH) connected with a linker short enough to prevent interaction of these two domains, for example a linker of about five amino acids. VL and VH domains encoded on the same polypeptide chain will form a dimer because the linker between the VL and VH is too short to form a single chain variable region fragment. Thus, the result is a diabody comprising two antigen-binding sites. If the VL and VH domains directed against two different antigens (a and b) are expressed simultaneously as combinations of VLa-VHb and VLb-VHa, connected with a linker of about five residues, they can be secreted as a bispecific diabody.

scFv or scFv antibody fragments contain the VH and VL regions of an antibody, and these regions exist on a single polypeptide chain (Huston, J. S. et al., Proc. Natl. Acad. Sci. U.S.A. (1988) 85, 5879-5883). Generally, a scFv polypeptide further contains a polypeptide linker between the VH and VL regions, and therefore an scFv can form a structure necessary for antigen binding. See Pluckthun “The Pharmacology of Monoclonal Antibodies” Vol. 113 (Rosenburg and Moore eds. (Springer Verlag, New York) pp. 269-315, 1994) for a review on scFv. Linkers are not especially limited in the present invention, as long as they do not inhibit expression of antibody variable regions linked at their ends.

Such antibody fragments and minibodies can be prepared by treating antibodies with an enzyme, for example, papain or pepsin, or alternatively by constructing genes encoding the antibody fragments, introducing them into expression vectors, and then expressing the vectors in appropriate host cells (see, for example, Co, M. S. et al., J. Immunol. (1994) 152, 2968-2976; Better, M. and Horwitz, A. H., Methods Enzymol. (1989) 178, 476-496; Pluckthun, A. and Skerra, A., Methods Enzymol. (1989) 178, 497-515; Lamoyi, E., Methods Enzymol. (1986) 121, 652-663; Rousseaux, J. et al., Methods Enzymol. (1986) 121, 663-669; Bird, R. E. and Walker, B. W., Trends Biotechnol. (1991) 9, 132-137).

Modified antibodies include, for example, antibodies conjugated with various molecules such as polyethylene glycol (PEG), and antibodies conjugated with cytotoxic substances, endotoxins, or radioactive substances. The substances to be conjugated with the modified antibodies of the present invention are not limited. Such modified antibodies can be obtained by chemically modifying prepared antibodies, and these methods are already established in the art.

The origin of antibodies of the present invention is not limited. The antibodies may be human, mouse, or rat antibodies. In addition, the antibodies may be genetically altered antibodies, such as chimeric or humanized antibodies.

Methods for preparing human antibodies are already known. For example, human antibodies of interest can be obtained by using an antigen of interest to immunize transgenic animals that have the entire repertoire of human antibody genes (see WO 93/12227, WO 92/03918, WO 94/02602, WO 94/25585, WO 96/34096, and WO 96/33735).

Genetically altered antibodies can be produced by conventional methods. Specifically, for example, chimeric antibodies comprise the variable regions of heavy and light chains of antibodies from an immunized animal, and the constant regions of heavy and light chains of a human antibody. Chimeric antibodies can be obtained by ligating DNAs that encode the variable regions of an antibody derived from an immunized animal with DNAs encoding the constant regions of a human antibody, inserting the ligated DNA into an expression vector, and then introducing the construct into a host.

Humanized antibodies are altered antibodies also referred to as “reshaped human antibodies”. Humanized antibodies can be constructed by transplanting the CDR of an antibody derived from an immunized animal to the CDR of a human antibody. This common gene recombination technique is also known.

Specifically, a DNA sequence designed such that the framework region (FR) of a human antibody is linked with a CDR of a mouse antibody is synthesized by PCR, using several oligonucleotides prepared to comprise overlapping portions at their ends. The synthesized DNA is ligated to a DNA that encodes the constant region of a human antibody, and is then inserted into an expression vector. The vector construct is introduced into a host to produce a humanized antibody (see EP 239400 and WO 96/02576). The human antibody FR to be ligated with CDR is selected so that the CDR forms an adequate antigen-binding domain. If required, some amino acids in the framework region of the antibody variable region may be replaced with other amino acids so that the CDR of the reshaped human antibody forms an adequate antigen-binding domain (Sato, K. et al., Cancer Res. (1993) 53, 851-856).

The methods for preparing agonist antibodies of the present invention are not limited, and the antibodies may be prepared by any method. For example, when preparing an agonist antibody against a heteromeric receptor comprising two chains (A and B chains), animals are immunized with each of the two chains (A and B) constituting the receptor, and more than one anti-A chain antibody and more than one anti-B chain antibody are obtained. Then, bispecific antibodies are prepared, which comprise H and L chains of an anti-A chain antibody, and H and L chains of an anti-B chain antibody. As described above, a number of anti-A chain antibodies and anti-B chain antibodies can be obtained, and thus it is preferable to prepare as many bispecific antibodies as possible, comprising different combinations of anti-A chain and anti-B chain antibodies. After preparing the bispecific antibodies, antibodies with agonist activity are selected.

In an embodiment of the present invention, antibodies of the present invention are bispecific antibodies. The bispecific antibodies can be prepared by known methods, such as by fusing antibody-producing hybridomas or introducing antibody expression vectors to cells. For example, animals are immunized with either an A or B chain of a receptor, mRNA is extracted from their spleen cells, and then the variable regions of L and H chains are recovered by RT-PCR using primers that correspond to the variable regions containing CDRs. Single-chain Fv (scFv) is synthesized by assembly PCR to construct a phage library. Antigen-binding antibody clones are concentrated and cloned by panning. The single-chain variable region (scFv) is then inserted between CH1-hinge-CH2-CH3 and the signal sequence for animal cells to prepare a scFv-CH1-Fc expression vector. Bispecific antibodies can be obtained by introducing a vector encoding anti-A chain antibody and a vector encoding anti-B chain antibody into the same cell, and then expressing the antibody.

Antibodies having agonist activity can be selected, for example, by a method described below:

(1) Determining whether or not a cell that is growing factor-dependently will grow in the same way when an antibody is added during cell culture as when a factor is added. If the cell grows, the test multispecific antibody is determined to have agonistic function.

(2) Determining whether or not a cell line with intrinsic factor-dependent activities (not limited to growth) shows the same reaction when an antibody is added during cell culture as when a factor is added. If the cell line shows the same reaction as for a factor, the antibody is determined to have agonistic function.

The receptors for which the antibodies of the present invention act as agonists are usually expressed on the cell surface of the above-described cells. These cells mediate cell growth signals by binding with the ligands of those receptors (for example, agonistic antibodies). Thus, the cells used in the methods described above are preferably cells that can proliferate receptor ligand (factor)-dependently (cells with factor-dependent proliferation). Preferably, on binding with a ligand, the above-described receptors usually mediate cell growth signals. However, when the receptors are of a type that do not mediate cell growth signals, they can be used in the above-described methods as so-called “chimeric receptors”, by fusing with receptors of a type that do mediate cell growth signals. These chimeric receptors mediate cell growth signals on binding with a ligand. Receptors suitable for constructing chimeric receptors by fusion with the above-described receptors are not especially limited, as long as they are of a type that mediates cell growth signals. Typically the receptor is a transmembrane protein, and more preferably a receptor whose extracellular portion comprises a ligand receptor chain and whose intracellular portion is a receptor chain. Specifically, such receptors include G-CSF receptor, mpl, neu, GM-CSF receptor, EPO receptor, c-Kit, and FLT-3. Specific examples of preferred cells with factor-dependent proliferation include BaF3, a factor-dependently proliferating cell that expresses a chimeric receptor whose extracellular portion comprises a ligand receptor chain and whose intracellular portion is the G-CSF receptor chain. In addition, cells that can be used in the methods described above include, for example, NFS60, FDCP-1, FDCP-2, CTLL-2, DA-1, and KT-3.

Furthermore, methods for selecting antibodies having agonist activity include methods that use various types of quantitative and/or qualitative change as an indicator. For example, such methods may use cell-free assays, cell-based assays, tissue-based assays, and biological assays as indicators.

In cell-free assays, enzyme reaction and quantitative and/or qualitative changes of protein, DNA, or RNA can be used as indicators. The enzyme reaction includes, for example, amino acid transfer reactions, sugar transfer reactions, dehydration reactions, dehydrogenation reactions, and substrate cleavage reactions. In addition, protein phosphorylation, dephosphorylation, dimerization, multimerization, degradation, dissociation, and the like, and DNA and RNA amplification, cleavage and extension can also be used as indicators. For example, protein phosphorylation downstream of a signal pathway may be used as a detection indicator.

Indicators that can be used in cell-based assays include alterations in cell phenotype, for example, quantitative and/or qualitative alterations in a product, and alterations in cell growth activity, cell count, morphology, and cell properties. Products include secretory proteins, surface antigens, intracellular proteins, and mRNAs. Morphological alterations include process formation and/or alterations in the number of processes, the flatness, the degree of extension/aspect ratio, cell size, intracellular structure, heterogeneity/homogeneity of cell populations, and cell density. These morphological alterations can be observed under a microscope. Alterations in cell properties include anchor dependency, cytokine-dependent response, hormone dependency, drug resistance, cell motility, cell migration activity, pulsatility, and alterations in intracellular substances. Cell motility includes cell infiltration activity, and cell migration activity. Alterations of intracellular substances include, for example, enzymatic activities, mRNA content, the content of signaling molecules such as Ca²⁺ and cAMP, and the content of intracellular proteins. Alterations in cell growth activity, induced by stimulation of the receptor, can be used as an indicator for a cell membrane receptor.

In tissue-based assays, functional alterations in tissues used can be used as detection indicators. Biological assays can be conducted using alterations in tissue weight, blood system (such as alterations in blood cell populations), protein amount, enzymatic activities, and electrolyte amount, and also alterations in the circulating system such as alterations in blood pressure and heart rate.

Methods for measuring such detection indicators are not specifically limited, and absorbance, luminescence, coloration, fluorescence, radioactivity, fluorescence polarization, surface plasmon resonance signals, time resolution fluorescence, mass, absorption spectrum, light scattering, fluorescence resonance energy transfer, and the like can be used. These measurement methods are known to those skilled in the art, and can be properly selected depending on the aim.

For example, absorption spectra can be measured using a conventional photometer, plate reader, or the like; luminescence can be measured using a luminometer or the like; and fluorescence can be measured using a fluorometer or the like. Mass can be determined using a mass spectrometer. Radioactivity can be determined using devices such as gamma counters or the like, depending the type of radioactivity. Fluorescence polarization can be measured using BEACON (TaKaRa Shuzo), and surface plasmon resonance signals can be measured using BIACORE. Time resolution fluorescence and fluorescence resonance energy transfers can be measured using ARVO. In addition, flow cytometers can also be used for measurement. Two or more detection indicators may be measured by one of the measurement methods described above. If they can be simply performed, two or more measurements may be carried out simultaneously and/or continuously to measure even more detection indicators. For example, fluorescence and fluorescence resonance energy transfer can be measured simultaneously using a fluorometer.

Antibodies against a receptor can be obtained by methods known to those skilled in the art. For example, antibodies can be prepared by immunizing animals with an antigen. The antigens for immunizing animals include complete antigens with immunogenicity, and incomplete antigens (including haptens) without immunogenicity. In the present invention, receptors on which an agonist antibody of the present invention is predicted to act as a ligand are used as the above-described antigens (immunogens). The receptors of the present invention are not specifically limited, but are preferably heterodimeric receptors. Animals used for immunization include, for example, mice, hamsters, and Rhesus monkeys. These animals can be immunized with antigens using methods known to those skilled in the art. For example, in a general method, a mammal is intraperitoneally or subcutaneously injected with a sensitized antigen. Specifically, a sensitized antigen is diluted to an appropriate volume with PBS (phosphate-buffered saline), physiological saline, or the like, and the suspended antigen is combined with an adequate amount of a conventional adjuvant, for example, Freund's complete adjuvant, as required. After emulsification, the resulting emulsion is given to mammals several times every 4 to 21 days. After the mammals are immunized as described above, an increased level of a desired antibody in sera is confirmed, and immune cells are collected from the mammals.

In the present invention, the variable regions of antibody L and H chains are preferably recovered from immunized animals or their cells. This procedure can be achieved by methods well known to those skilled in the art. Animals immunized with an antigen express antibodies to that antigen in their spleen cells especially. Thus, for example, mRNAs can be prepared from the spleen cells of immunized animals, and then the variable regions of the L and H chains can be recovered by RT-PCR using primers that correspond to the CDRs of the animals. The term “CDR” refers to the three regions (CDR1, CDR2, and CDR3) which complementarily and directly bind antigens, and which are present in hypervariable regions within the antibody variable regions. Primers corresponding to a CDR include, for example, primers corresponding to framework regions with lower variety than the CDR; and primers corresponding to signal sequences and the CH1 and CL1 portions. Alternatively, lymphocytes can be immunized in vitro. Then, DNAs encoding antibodies can be isolated from the spleen or lymphocytes of the immunized animals using conventional methods, for example, by a method using nucleotide probes or the like capable of specifically binding genes that encode the heavy and light chains of the antibodies.

Receptors for use as immunogens may be whole proteins constituting the receptor, or a partial peptide derived from the protein. In some cases, an immunogen for immunizing animals may be a soluble antigen prepared by binding an antigen to another molecule. Alternatively, fragments of the antigen may be used, as required. When a transmembrane molecule such as a receptor is used as an antigen, fragments of the molecule are preferably used (for example, the extracellular domain of the receptor). Alternatively, cells expressing a transmembrane molecule on their surface can be used as immunogens. Such cells may be natural cells (tumor cell lines and the like) or cells modified by genetic recombination techniques to express a transmembrane molecule.

Prepared antibodies can be purified to homogeneity. Antibodies can be isolated and purified by conventional methods for isolating and purifying proteins. For example, antibodies can be isolated and purified by appropriately selecting or combining methods that include, but are not limited to chromatographic columns for affinity chromatography or such, filtration, ultrafiltration, salting out, dialysis, SDS-polyacrylamide gel electrophoresis, and isoelectrofocusing (Antibodies: A Laboratory Manual. Ed Harlow and David Lane, Cold Spring Harbor Laboratory, 1988). Columns used in affinity chromatography include protein A columns and protein g columns. For example, protein A columns include Hyper D, POROS, and Sepharose F.F. (Pharmacia).

For example, when the antibodies of the present invention are antibodies with agonist activity to a type-I interferon receptor comprising AR1 and AR2 chains, they preferably have a structure comprising variable regions of an anti-AR1 chain antibody as well as variable regions of an anti-AR2 chain antibody. For example, the antibody includes, but is not limited to, those containing any of the following anti-AR1 chain antibody variable regions, and any of the following anti-AR2 chain antibody variable regions:

Variable regions of anti-AR1 chain antibodies: AR1-41 and AR1-24

Variable regions of anti-AR2 chain antibodies: AR2-37, AR2-11, AR2-13, AR2-45, AR2-22, AR2-43, AR2-40, AR2-14, AR2-44, AR2-33, and AR2-31

The amino acid sequences of VH and VL of each of the variable regions listed above are shown in SEQ ID NOs: 1-26 (Table 1 shows the relationship between SEQ ID NOs and the VH and VL of each variable region).

TABLE 1

SEQ ID NO:

| | | |
|---|---|---|
| Variable region | VH | VL |

| | | |
|---|---|---|
| AR1-41 | 1 | 2 |
| AR1-24 | 3 | 4 |
| AR2-37 | 5 | 6 |
| AR2-11 | 7 | 8 |
| AR2-13 | 9 | 10 |
| AR2-45 | 11 | 12 |
| AR2-22 | 13 | 14 |
| AR2-43 | 15 | 16 |
| AR2-40 | 17 | 18 |
| AR2-14 | 19 | 20 |
| AR2-44 | 21 | 22 |
| AR2-33 | 23 | 24 |
| AR2-31 | 25 | 26 |

When the anti-AR1 chain antibody is AR1-24, the partner anti-AR2 chain antibody is preferably any one of AR2-13, AR2-31, and AR2-44. When the anti-AR1 chain antibody is AR1-41, the partner anti-AR2 chain antibody is preferably any one of AR2-11, AR2-13, AR2-14, AR2-22, AR2-33, AR2-37, AR2-40, AR2-43, AR2-44, and AR2-45. AR2-13 and AR2-44 can be partnered with either antibody AR1-41 or AR1-24. Such antibody pairs are also included in the present invention.

In the present invention, it is not necessary for the antibody comprising the variable region described above to contain the full-length sequence of the variable region. As long as it retains the CDR sequence, the FR sequence may be appropriately modified at the time of antibody humanization or such.

Thus, the antibodies of the present invention include antibodies comprising amino acid sequences that correspond to CDRs in the variable region described above.

Table 2 shows the numbers of the amino acids that correspond to the CDRs in each SEQ ID NO for the above-described variable regions.

TABLE 2

| | | | |
|---|---|---|---|
| SEQ | | SEQ | |
| ID | | ID | |
| NO: | Amino acid number | NO: | Amino acid number |

| | | | | | | | |
|---|---|---|---|---|---|---|---|
| VH | CDR1 | CDR2 | CDR3 | VL | CDR1 | CDR2 | CDR3 |

| | | | | | | | | |
|---|---|---|---|---|---|---|---|---|
| AR1-41 | 1 | 31-35 | 50-66 | 99-109 | 2 | 24-34 | 50-56 | 89-97 |
| AR1-24 | 3 | 31-35 | 50-66 | 99-108 | 4 | 24-34 | 50-56 | 89-97 |
| AR2-37 | 5 | 31-35 | 50-66 | 99-106 | 6 | 24-39 | 55-61 | 94-102 |
| AR2-11 | 7 | 31-35 | 50-66 | 99-108 | 8 | 24-39 | 55-61 | 94-102 |
| AR2-13 | 9 | 31-35 | 50-66 | 99-107 | 10 | 24-39 | 55-61 | 94-102 |
| AR2-45 | 11 | 31-35 | 50-66 | 99-107 | 12 | 24-39 | 55-61 | 94-102 |
| AR2-22 | 13 | 31-35 | 50-66 | 99-106 | 14 | 24-39 | 55-61 | 94-102 |
| AR2-43 | 15 | 31-35 | 50-66 | 99-106 | 16 | 24-39 | 55-61 | 94-102 |
| AR2-40 | 17 | 31-35 | 50-66 | 99-106 | 18 | 24-39 | 55-61 | 94-102 |
| AR2-14 | 19 | 31-35 | 50-66 | 99-108 | 20 | 24-39 | 55-61 | 94-102 |
| AR2-44 | 21 | 31-35 | 50-66 | 99-108 | 22 | 24-39 | 55-61 | 94-102 |
| AR2-33 | 23 | 31-35 | 50-66 | 99-108 | 24 | 24-39 | 55-61 | 94-102 |
| AR2-31 | 25 | 31-35 | 50-66 | 99-106 | 26 | 24-39 | 55-61 | 94-102 |

When preparing full-length antibodies using the variable regions disclosed herein, the type of constant region is not limited, and constant regions known to those skilled in the art can be used. For example, it is possible to use the constant regions described in “Sequences of proteins of immunological interest”, (1991), U.S. Department of Health and Human Services. Public Health Service National Institutes of Health; and “An efficient route to human bispecific IgG”, (1998). Nature Biotechnology vol. 16, 677-681”.

Since the antibodies of the present invention have the agonist activity, they can be used as effective pharmaceutical agents for diseases caused by reduced activity (function) of the receptor on which the antibody acts. Specifically, the present invention provides pharmaceutical compositions that comprise an antibody of the present invention as an active ingredient. For example, when an antibody of the present invention has agonist activity to a cytokine receptor, it may have cytokine-like activity. Thus, the antibody is expected to be a pharmaceutical (pharmaceutical composition) with antiviral activity, antitumor activity, or cell growth/differentiation regulating activity.

Pharmaceutical compositions used for therapeutic or preventive purposes, which comprise antibodies of the present invention as active ingredients, may be formulated by mixing as necessary with suitable pharmaceutically acceptable carriers and solvents that are non-reactive to the antibodies. For example, sterilized water, saline, stabilizers, vehicles, antioxidants (e.g. ascorbic acid), buffers (e.g. phosphate, citrate, other organic acids), preservatives, detergents (e.g. PEG, Tween), chelating agents (e.g. EDTA), and binders may be used. Alternatively, the pharmaceuticals may comprise other low molecular weight polypeptides, proteins such as serum albumin, gelatin and immunoglobulins, amino acids such as glycine, glutamine, asparagine, arginine, and lysine, carbohydrates and sugars such as polysaccharides and monosaccharides, and sugar alcohols such as mannitol and sorbitol. When prepared as an aqueous solution for injection, saline and isotonic solutions comprising glucose and other adjunctive agents such as D-sorbitol, D-mannose, D-mannitol, and sodium chloride may be used. In addition, appropriate solubilizing agents such as alcohols (e.g. ethanol), polyalcohols (e.g. propylene glycol, PEG), and non-ionic detergents (e.g. polysorbate 80, HCO-50) may be used in combination.

If necessary, antibodies of the present invention may be encapsulated in microcapsules (microcapsules made of hydroxycellulose, gelatin, polymethylmethacrylate, and the like), and made into components of colloidal drug delivery systems (liposomes, albumin microspheres, microemulsions, nano-particles, and nano-capsules) (refer to, for example, “Remington's Pharmaceutical Science 16th edition”, Oslo Ed. (1980)). Moreover, methods for making controlled-release drugs are known, and these can be applied to the antibodies of the present invention (Langer et al., J. Biomed. Mater. Res. 15: 167-277 (1981); Langer, Chem. Tech. 12: 98-105 (1982); U.S. Pat. No. 3,773,919; EP Patent Application No. 58,481; Sidman et al., Biopolymers 22: 547-556 (1983); EP: 133,988).

The dose of a pharmaceutical composition of the present invention depends on the type of dosage form, administration method, patient's age, weight, symptoms, disease progression, and other factors, and ultimately should be properly determined by physicians. Typically, 0.1 to 2000 mg/day can be administered orally to an adult once or several times. More preferably the dose ranges from 1 to 1000 mg/day, still more preferably from 50 to 500 mg/day, most preferably from 100 to 300 mg/day. The dose varies depending on a patient's weight and age, the method of administration, and the like, however, it can be properly selected by those skilled in the art. The period of administration is preferably properly selected depending on each patient's course of treatment and the like.

In addition, genes encoding antibodies of the present invention may be used for gene therapy by cloning into gene therapy vectors. Such vectors can be administered by direct injection using naked plasmids, and also by packaging in liposomes, producing a variety of viral vectors such as retrovirus vectors, adenovirus vectors, vaccinia virus vectors, poxvirus vectors, adenoassociated virus vectors, and HVJ vector (Adolph, “Virus Genome Methods”, CRC Press, Florida (1996)), or by coating onto carrier beads such as colloidal gold particles (e.g. WO93/17706). However, any method can be used for administration as long as the antibodies are expressed in vivo and exercise their function. Preferably, a sufficient dose may be administered by a suitable parenteral route (such as intravenous, intraventricular, subcutaneous or percutaneous injection, or injection into adipose tissues or mammary glands, inhalation, intramuscular injection, infusion, gas-induced particle bombardment (using electron guns and such), or through the mucosa (for example, by nose drops)). Alternatively, genes encoding antibodies of the present invention may be administered into, for example, blood cells and bone marrow cells ex vivo using liposome transfection, particle bombardment (U.S. Pat. No. 4,945,050), or viral infection, and these cells may be reintroduced into animals.

### BRIEF DESCRIPTION OF THE DRAWINGS

FIG. 1 shows the growth-suppressing activities of antibodies in Daudi cells. Dose-dependent agonist activity could be confirmed.

FIG. 2 shows the ISRE activation ability of antibodies in pISRE-Luc-introduced K562 cells. The results of measuring the luciferase activity of AR1-41/AR2-13 are shown. Open squares indicate IFN-α2a, and closed circles indicate the bispecific antibody AR1-41/AR2-13.

FIG. 3 shows the ISRE activation ability of antibodies in pISRE-Luc-introduced K562 cells. Assay results for the luciferase activity of AR1-41/AR2-13 are shown. Open squares indicate IFN-α2a, and closed circles indicate the bispecific antibody AR1-41/AR2-14.

FIG. 4 shows the ISRE activation ability of antibodies in pISRE-Luc-introduced K562 cells. Assay results for the luciferase activity of AR1-41/AR2-13 are shown. Open squares indicate IFN-α2a, and closed circles indicate the bispecific antibody AR1-24/AR2-13.

FIG. 5 shows the ISRE activation ability of antibodies in pISRE-Luc-introduced K562 cells. Assay results for the luciferase activity of AR1-41/AR2-13 are shown. Open squares indicate IFN-α2a, and closed circles indicate the bispecific antibody AR1-24/AR2-31.

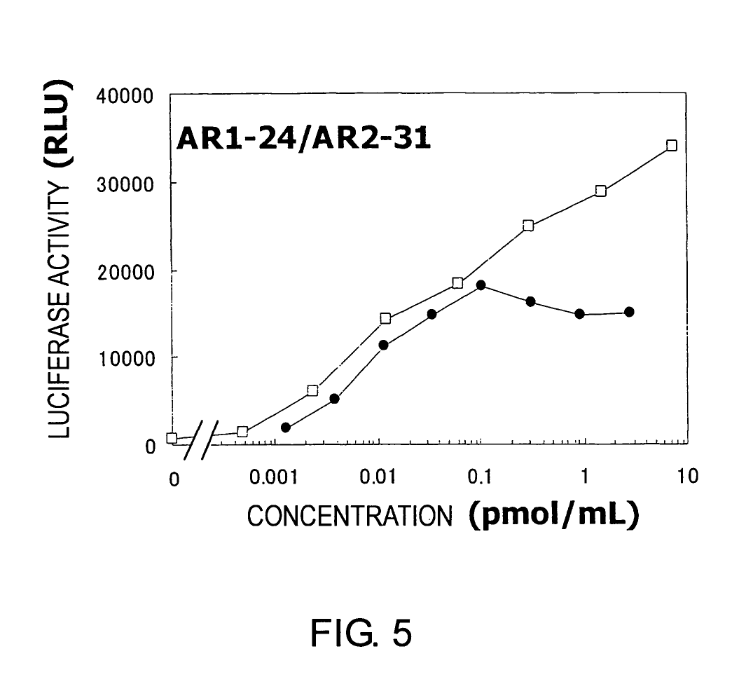

### BEST MODE FOR CARRYING OUT THE INVENTION

Herein below, the present invention will be specifically described using Examples, however, it is not to be construed as being limited thereto.

### EXAMPLE 1

### Antigens and Immunization

CHO cells were transfected with an expression vector for a solubilized receptor comprising the extracellular domains of human AR1 and AR2 attached with FLAG or His6 tag at their C termini. The receptor was purified from the culture supernatant using an affinity column. An expression vector for the chimeric molecule that was formed between G-CSF receptor and the extracellular domain of human AR1 was introduced into mouse pro-B cell line BaF3 to establish a receptor-overexpressing cell line. Likewise, another cell line overexpressing the chimeric molecule between G-CSF receptor and the extracellular domain of human AR2 was established. Cells of each cell line were injected to the peritoneal cavity of BALB/c mice for immunization. Three days before spleen excision, AR1 His or AR2His was intravenously injected to the mice.

### EXAMPLE 2

### Isolation of Antibodies from a scFv-Presenting Library

(a) Panning of a Phage Library

PolyA(+) RNA was extracted from the spleens of immunized mice, and scFv was then synthesized by RT-PCR. A plasmid library was constructed that expresses scFv as a fusion protein with the product of the f1 phage gene 3 (J. Immun. Methods, 201, (1997), 35-55). An E. coli library (2×10⁹ cfu) was inoculated to 50 ml of 2×YTAG (2×TY containing 100 μg/ml ampicillin and 2% glucose), and the culture was incubated at 37° C. until OD₆₀₀ reached 0.4-0.5. 4×10¹¹ helper phage VCSM13 were added, and the culture was allowed to stand for 15 minutes at 37° C. to be infected. 450 mL of 2×YTAG and 25 μl of 1 mol/l IPTG were added to the culture, and incubated at 26° C. for ten hours. The culture supernatant was collected by centrifugation. 100 ml of PEG-NaCl (10% polyethylene glycol 8000 and 2.5 mol/l NaCl) was combined with the supernatant, and allowed to stand at 4° C. for 60 minutes. Phages were precipitated by centrifugation at 10,800×g for 30 minutes. The precipitate was suspended in 40 ml of water, and mixed with 8 ml of PEG-NaCl, then allowed to stand at 4° C. for 20 minutes. Phages were precipitated by centrifugation at 10,800×g for 30 minutes. The precipitate was suspended in 5 ml of PBS. AR1FLAG and AR2FLAG were labeled with biotin using No-Weigh Premeasured NHS-PEO4-Biotin Microtubes (Pierce). 100 pmol of biotin-labeled AR1FLAG or AR2FLAG was added to the phage library. The antigen was contacted with the phages for 60 minutes. 600 μl of Streptavidin MagneSphere (Promega), which was washed with 5% M-PBS (PBS containing 5% skimmed milk), was added, and allowed to bind to the antigen for 15 minutes. The beads were washed three times with 1 ml of PBST (PBS containing 0.1% Tween-20) and then three times with PBS. The beads were suspended in 0.8 ml of 0.1 mol/l glycine/HCl (pH 2.2) for five minutes to elute the phages. The collected phage solution was neutralized by adding 45 μl of 2 mol/l Tris. The phage solution was added to 10 ml of XL1-Blue culture during the logarithmic growth phase (OD₆₀₀ of 0.4-0.5), and allowed to stand at 37° C. for 30 minutes for infection. The culture was spread on 2×YTAG plates, and then incubated at 30° C. Colonies were collected and inoculated to 2×YTAG, and then incubated at 37° C. until OD₆₀₀ reached 0.4-0.5. 5 μl of 1 mol/l IPTG and 10¹¹ pfu of helper phage (VCSM13) were added to 10 ml of the culture liquid, and allowed to stand at 37° C. for 30 minutes. The bacterial cells were collected by centrifugation, and then re-suspended in 100 ml of 2×YTAG containing 25 μg/ml kanamycin. The suspension was incubated at 30° C. for ten hours. The culture supernatant was collected by centrifugation and mixed with 20 ml PEG-NaCl, then allowed to stand at 4° C. for 20 minutes. Phages were precipitated by centrifugation at 10,800×g for 30 minutes, and then re-suspended in 2 ml of PBS. The suspension was used in subsequent panning experiments. In the second-round panning, the beads were washed five times with PBST and then five times with PBS. The eluted phages were infected to E. coli cells, and clones producing AR-binding phages were selected from the resultant E. coli cells.

(b) Phage ELISA

The single colonies described above were inoculated to 150 μl of 2×YTAG and cultured at 30° C. overnight. 51 μl of the cultures was inoculated to 500 μl of 2×YTAG, and incubated at 37° C. for two hours. Then 100 μl of 2×YTAG containing 2.5×10⁹ pfu of helper phage and 0.3 μl of 1 mol/l IPTG was added to the cultures, and allowed to stand at 37° C. for 30 minutes. The cultures were then incubated at 30° C. overnight, and their culture supernatant was subjected to ELISA. StreptaWell 96-well microtiter plates (Roche) were coated overnight with 100 μl of PBS containing 1.0 μg/ml biotin-labeled AR1FLAG or AR2FLAG The plates were washed with PBST to remove free antigens, and then blocked overnight with 200 μl of 2% M-PBS. 2% M-PBS was removed and the culture supernatant was added to the plates. The plates were allowed to stand for 40 minutes to bind with the antibodies. After the plates were washed, bound phages were detected using HRP-conjugated anti-M13 antibody (Amersham Pharmacia Biotech) diluted with 2% M-PBS and BM blue POD substrate (Roche). The reaction was stopped by adding sulfuric acid, and absorbance was determined at 450 nm.

(c) Sequencing and Clone Selection

The scFv regions were amplified from phage solutions of the clones that were positive in ELISA, by PCR using the primers PBG3-F1 (5′-CAGCTATGAAATACCTATTGCC-3′/SEQ ID NO: 27) and PBG3-R1 (5′-CTTTTCATAATCAAAATCACCGG-3′/SEQ ID NO: 28), and the nucleotide sequences were determined. 20 μl of PCR solution containing 1 μl of phage solution, 2 μl of 10×KOD Dash buffer, 0.5 μL each of 10 μmol/l primers, and 0.3 μl of KOD Dash polymerase (TOYOBO, 2.5 U/μl) was prepared, and subjected to 30-cycle amplification of 96° C. for 10 seconds, 55° C. for 10 seconds, and 72° C. for 30 seconds with Perkin Elmer 9700. After PCR, 3 μl of ExoSAP-IT (Amersham) was added to 5 μl of the reaction solution, and incubated at 37° C. for 20 minutes, and subsequently at 80° C. for 15 minutes. The sample was reacted with BigDye Terminator Cycle Sequencing kit (Applied Biosystems) using the primer PBG3-F2 (5′-ATTGCCTACGGCAGCCGCT-3′/SEQ ID NO: 29) or PBG3-R2 (5′-AAATCACCGGAACCAGAGCC-3′/SEQ ID NO: 30), and then electrophoresed in an Applied Biosystems PRISM 3700 DNA Sequencer. 45 clones were selected for both anti-AR1 and anti-AR2, comprising different CDR3 amino acid sequences as deduced from the nucleotide sequences.

### EXAMPLE 3

### Expression of Bispecific Antibodies

The expression vector pCAGGss-g4CH-hetero-IgG4 was constructed to express scFv-CH1-Fc. scFv can be inserted into the vector at the SfiI site, between intron-CH1-Fc (human IgG4 cDNA) and the human signal sequence driven by the CAGG promoter. With reference to IgG1 knobs-into-holes technology (Protein Engineering vol. 9, 617-621, 1996, Nature Biotechnology vol. 16, 677-681, 1998), the amino acids of the CH3 of IgG4 were substituted in order to express heteromeric molecules. Type A are Y349C and T366W mutants; and type B are E356C, T366S, L368A, and Y407V mutants. In addition, an amino acid was substituted in the hinge domain for both mutant types (from -ppcpScp- to -ppcpPcp-). Types A were constructed using the human IL-3 signal sequence, while types B were constructed using the human IL-6 signal sequence (pCAGG-IL3ss-g4CHPa and pCAGG-IL6ss-g4CHPb). PCR products, corresponding to the scFv regions of the clones selected based on their nucleotide sequences, were treated with SfiI. The products from the anti-AR1 clones were subcloned into pCAGG-IL3ss-g4CHPa, and those from the anti-AR2 clones were subcloned into pCAGG-IL3ss-g4CHPb. Expression vectors for each of the 2025 combinations of the 45 anti-AR1 clones and 45 anti-AR2 clones were transfected into HEK293 cells using lipofectamine 2000. The culture supernatants were collected three days after transfection.

### EXAMPLE 4

### Isolation of Bispecific Agonist Antibodies

(a) BaF3 Cell Growth Assay

BaF3-ARG was established by introducing murine BaF3 cells, which proliferate in a IL-3-dependent manner, with expression vectors for the chimeric molecules between the intracellular region of G-CSF receptor and the extracellular domains of AR1 and AR2. BaF3-ARG growth was dependant on IFNα. The cells were washed three times, and each well of a 96-well plate was inoculated with 1×10³ cells in 0.1 ml of sample-comprising culture medium. After incubating for four days, 10 μl of Cell Count Reagent SF (nacalai tesque) was added to the culture. The mixture was incubated at 37° C. for two hours, and then absorbance was determined at 450 nm.

(b) Growth Suppression Assay of Daudi Cells

Daudi cell is a human B cell line that is hypersensitive to IFN. Each well of a 96-well plate was inoculated with 6.25×10³ cells in 0.1 ml of sample-comprising culture medium. After incubating for four days, 10 μl of Cell Count Reagent SF (nacalai tesque) was added to the culture. The mixture was incubated at 37° C. for two hours, and then absorbance was determined at 450 nm.

(c) Sequences of Bispecific Agonist Antibodies

The amino acid sequences of variable regions of the antibodies selected through the screening described above are shown in SEQ ID NOs: 1-26. The correspondence between each antibody's name and SEQ ID NO is shown in Table 1 above.

(d) Reporter Gene Assay Using ISRE

100 μl of DMRIE-C (Invitrogen) was added to 3 ml of OPTI-MEM I and stirred. 40 μg of pISRE-Luc was added to the mixture. After stirring, the mixture was allowed to stand for 20 minutes at room temperature. The mixture was added to 2 ml of OPTI-MEM I containing 8×10⁶ cells of the human cell line K562. The cells were incubated at 37° C. for four hours. Then, 10 ml of 15% FCS-RPMI1640 was added to the culture, which was further incubated overnight. On the following day, the K562 cells were collected by centrifugation, and then re-suspended in 10.5 ml of 10% FCS-RPMI1640. 70 μl of the suspension was plated into each well of a 96-well flat-bottomed plate.

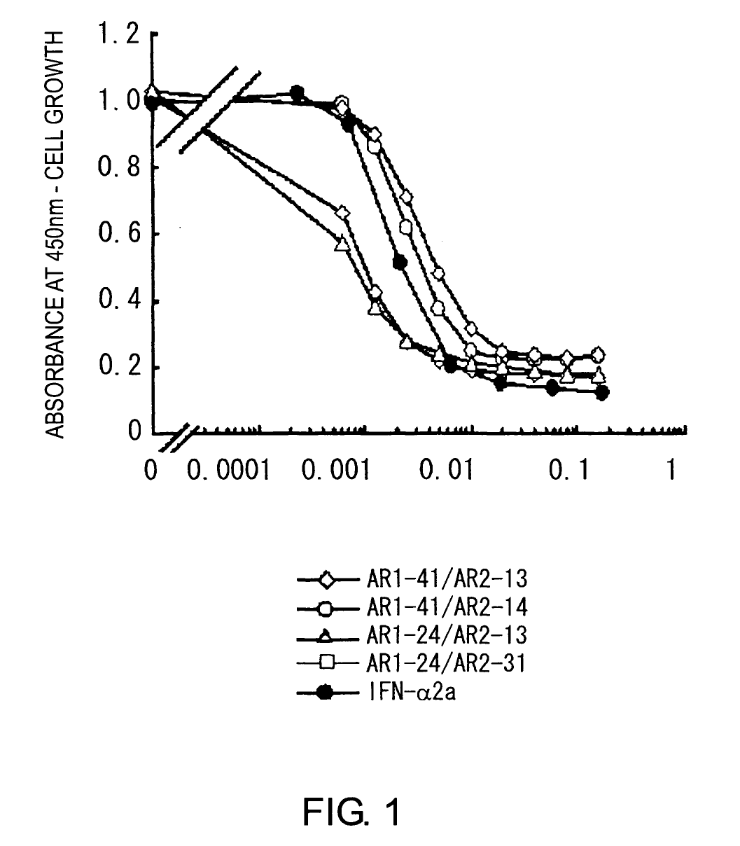

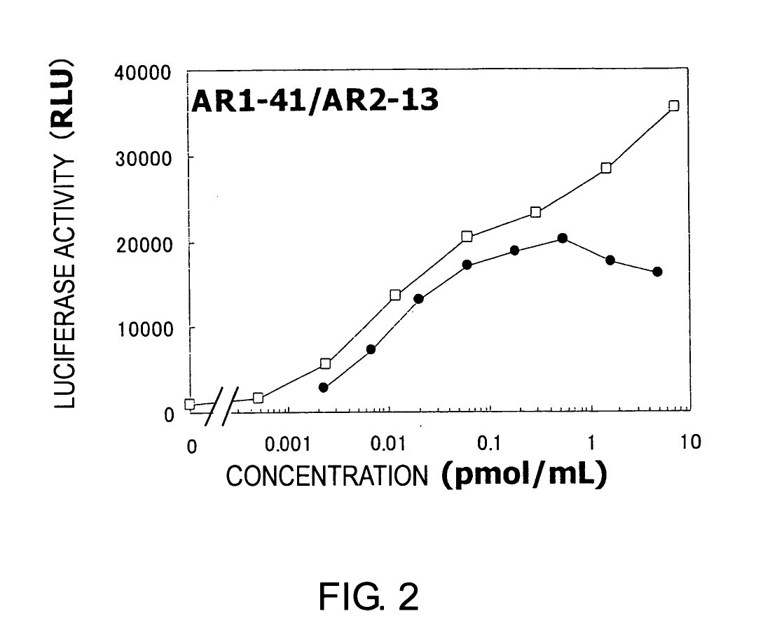

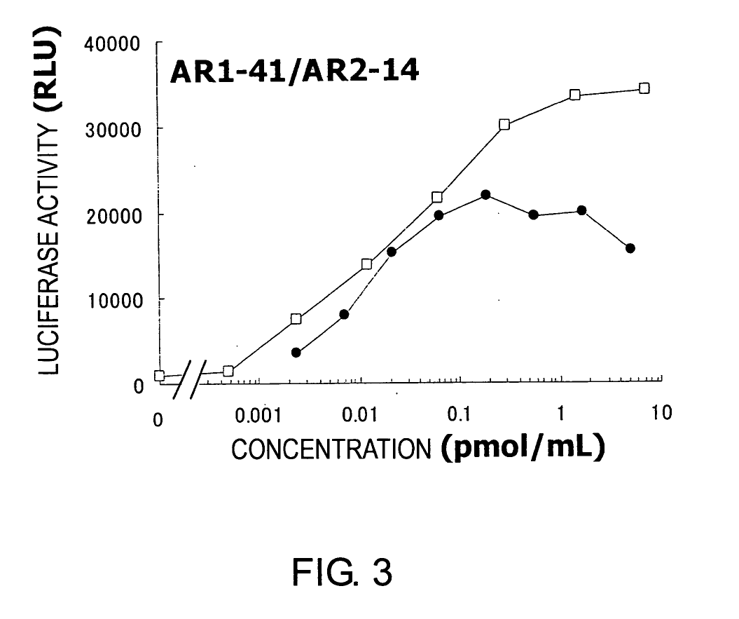

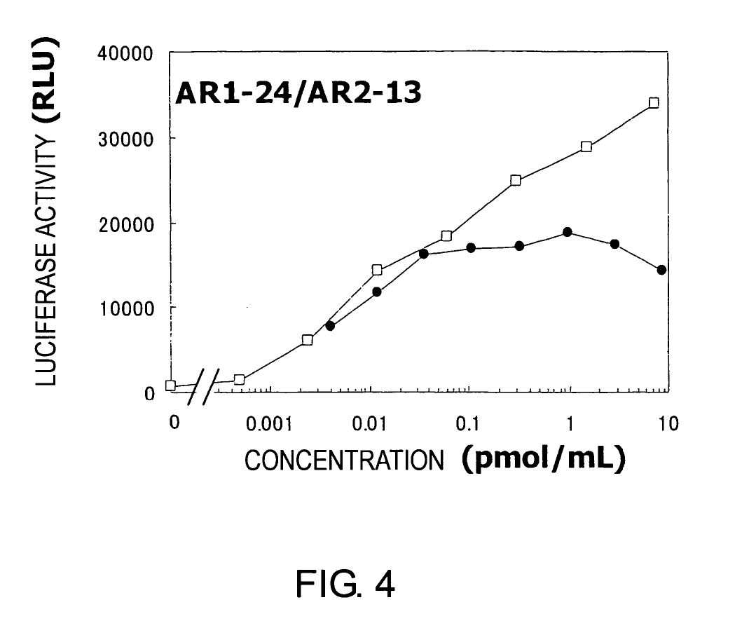

The concentration of bispecific scFv-CH in the supernatant of the antibody gene-introduced HEK293 culture was adjusted to be equivalent to 12.5 ng/mL IgG, and a five-fold dilution series was further prepared. 30 μl of the diluted solutions was added to each well containing the reporter plasmid-introduced cells. 30 μl of the five-fold dilution series of IFN-α2a was aliquoted into wells as a positive control. After incubation at 37° C. for 24 hours, 50 μl/ml of Bright-Glo Luciferase Assay System (Promega) was added, and the mixture was allowed to stand at room temperature for ten minutes. Luciferase activity was determined using Analyst HT (LJL) (FIG. 1, FIG. 2, FIG. 3, and FIG. 4).

### INDUSTRIAL APPLICABILITY

The present invention provides antibodies having agonist activity to a receptor comprising heterologous chains. The antibodies of the present invention are thought to be highly stable in blood and to have no antigenicity, and are therefore highly expected to be useful as pharmaceuticals.

1. An antibody having agonist activity to a receptor comprising a heterologous chain.

2. The antibody of claim 1, wherein the receptor is a cytokine receptor.

3. The antibody of claim 2, wherein the cytokine receptor is an interferon receptor.

4. The antibody of claim 3, wherein the interferon receptor is a type-I interferon receptor.

5. The antibody of claim 4, wherein the type-I interferon receptor comprises an AR1 chain and an AR2 chain.

6. The antibody of claim 1, wherein the receptor is a multimer.

7. The antibody of claim 6, wherein the multimer is a dimer.

8. The antibody of claim 1, wherein the antibody is a bispecific antibody.

9. The antibody of claim 5, comprising a variable region of an anti-AR1 chain antibody and a variable region of an anti-AR2 chain antibody.

10. The antibody of claim 9, comprising a variable region of an anti-AR1 chain antibody that comprises the amino acid sequences of (a), and a variable region of an anti-AR2 chain antibody that comprises the amino acid sequences of any one of (b1) to (b10):
(a) the amino acid sequence of SEQ ID NO: 1 as the H chain variable region, and the amino acid sequence of SEQ ID NO: 2 as the L chain variable region;
(b1) the amino acid sequence of SEQ ID NO: 7 as the H chain variable region, and the amino acid sequence of SEQ ID NO: 8 as the L chain variable region;
(b2) the amino acid sequence of SEQ ID NO: 9 as the H chain variable region, and the amino acid sequence of SEQ ID NO: 10 as the L chain variable region;
(b3) the amino acid sequence of SEQ ID NO: 19 as the H chain variable region, and the amino acid sequence of SEQ ID NO: 20 as the L chain variable region;
(b4) the amino acid sequence of SEQ ID NO: 13 as the H chain variable region, and the amino acid sequence of SEQ ID NO: 14 as the L chain variable region;
(b5) the amino acid sequence of SEQ ID NO: 23 as the H chain variable region, and the amino acid sequence of SEQ ID NO: 24 as the L chain variable region;
(b6) the amino acid sequence of SEQ ID NO: 5 as the H chain variable region, and the amino acid sequence of SEQ ID NO: 6 as the L chain variable region;
(b7) the amino acid sequence of SEQ ID NO: 17 as the H chain variable region, and the amino acid sequence of SEQ ID NO: 18 as the L chain variable region;
(b8) the amino acid sequence of SEQ ID NO: 15 as the H chain variable region, and the amino acid sequence of SEQ ID NO: 16 as the L chain variable region;
(b9) the amino acid sequence of SEQ ID NO: 21 as the H chain variable region, and the amino acid sequence of SEQ ID NO: 22 as the L chain variable region;
(b10) the amino acid sequence of SEQ ID NO: 1 as the H chain variable region, and the amino acid sequence of SEQ ID NO: 12 as the L chain variable region.

11. The antibody of claim 9, comprising the variable region of an anti-AR1 chain antibody that comprises the amino acid sequences of (a), and the variable region of an anti-AR2 chain antibody that comprises the amino acid sequences of any one of (b1) to (b3):
(a) the amino acid sequence of SEQ ID NO: 3 as the H chain variable region, and the amino acid sequence of SEQ ID NO: 4 as the L chain variable region;
(b1) the amino acid sequence of SEQ ID NO: 9 as the H chain variable region, and the amino acid sequence of SEQ ID NO: 10 as the L chain variable region;
(b2) the amino acid sequence of SEQ ID NO: 25 as the H chain variable region, and the amino acid sequence of SEQ ID NO: 26 as the L chain variable region;
(b3) the amino acid sequence of SEQ ID NO: 21 as the H chain variable region, and the amino acid sequence of SEQ ID NO: 22 as the L chain variable region.

12. A pharmaceutical composition comprising the antibody of claim 1 as an active ingredient.
